(19) United States
(12) Patent Application Publication
(10) Pub. No.: US 2006/0167298 A1
(43) Pub. Date: Jul. 27, 2006

(54) Sulfated diglyceride derivatives, salts thereof, and processes for their preparation

(76) Inventor: Milton J. Rosen, Great Neck, NY (US)

Correspondence Address: COZEN O'CONNOR, P.C., 1900 MARKET STREET, PHILADELPHIA, PA, 19103-3508, US

(21) Appl. No.: 11/040,234

(22) Filed: Jan. 21, 2005

(51) Int. Cl. C07C 305/02

(52) U.S. Cl. 558/31

(57) ABSTRACT

The present invention relates to novel sulfated derivatives of disubstituted glycerides, and to methods for their preparation. In some embodiments, the present invention provides compounds having the Formula Ia or Ib: 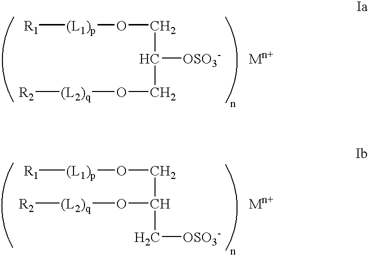 wherein the constituent variables are as defined herein.

### FIELD OF THE INVENTION

The present invention relates to sulfated diglycerides, and salts thereof, and to methods for their preparation.

### BACKGROUND OF THE INVENTION

Surfactants are active components in many different types of compositions, including detergents, emulsifiers, paints, adhesives, inks, wetting agents, and foaming agents. Surfactants are also widely employed in the pharmaceutical industry, for example as wetting agents in pharmaceutical compositions.

Various methods have been reported for the sulfation of glyceride monoester sulfates. (See U.S. Pat. No. 4,948,535). However, there have been no reports of the preparation of diglyceride sulfates.

Given the wide applicability of surfactants, there is a continuing need for novel surface active compounds. This invention is directed to these, as well as other, important ends.

### SUMMARY OF THE INVENTION

In some embodiments, the invention provides compounds having the Formula Ia or Ib: 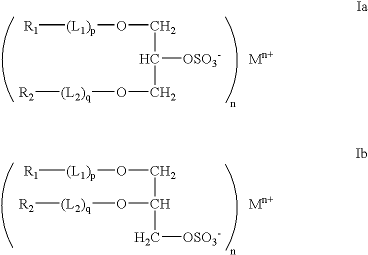
wherein:

M is a cation having charge n, for example an inorganic cation such as H⁺, Na⁺, K⁺ or NH₄⁺, or an organic cation such as primary, secondary, tertiary or quaternary ammonium, morpholinium or pyridinium ion;

n is 1, 2 or 3;

R₁ and R₂ are each independently C₁₋₂₅ alkyl, C₂₋₂₅ alkenyl, C₂₋₂₅ alkynyl, or C₂₋₂₅ polyunsaturated, each of which is optionally substituted with up to five substituents independently selected from the group consisting of halogen, optionally protected hydroxyl, C₁₋₁₂ alkoxy, C₁₋₂₄ alkoxyalkoxy, C₁₋₂₄ polyether, C₁₋₃ perhaloalkyl, C₁₋₃ perhaloalkoxy, or C₁₋₂₄ perhalopolyether;

L₁ and L₂ are each independently selected from the group consisting of —C(═O)— and —O—(C═O)—; and

p and q are each independently 0 or 1;

provided that R₁ and R₂ together contain at least six carbon atoms.

In some embodiments, p and q are each 1. In some further embodiments, p and q are each 0. In some embodiments, L₁ and L₂ are each —C(═O)—. In some further embodiments, p and q are each 1; and L₁ and L₂ are each —C(═O)—.

In some embodiments, R₁ and R₂ are each independently C₃₋₂₁ alkyl, or C₅₋₂₁ alkyl, or C₃₋₁₇ alkyl. In some embodiments, p and q are each 1; L₁ and L₂ are each —C(═O); and R₁ and R₂ are each independently C₃₋₂₁ alkyl, or R₁ and R₂ are each independently C₅₋₂₁ alkyl, or R₁ and R₂ are each independently C₃₋₁₇ alkyl. In some embodiments, p and q are each 1; L₁ and L₂ are each —C(═O); and R₁ and R₂ are each n-C₉H₁₉, or R₁ and R₂ are each n-C₁₀H₂₁, or R₁ and R₂ are each n-C₁₁H₂₃, or R₁ and R₂ are each n-C₁₂H₂₅.

In a further aspect, the invention provides processes for the sulfation of a disubstituted glyceride, the process comprising reacting the disubstituted glyceride with a sulfating agent in the presence of a catalyst for a time and under conditions effective to form a disubstituted glyceride sulfate, or a salt thereof; wherein the sulfating agent includes or consists of sulfamic acid, or a halosulfonic acid.

In some embodiments, the sulfating agent includes or consists of sulfamic acid, and the catalyst includes or consists of a sulfamate salt, for example ammonium sulfamate. In other embodiments, the sulfating agent includes or consists of sulfamic acid, and the catalyst includes or consists of an amine, for example a secondary amine, for example a cyclic aliphatic amine optionally containing up to three heteroatoms selected from O, N and S, such as morpholine, or an amide, such as urea or dicyandiamide.

In some embodiments, the sulfating agent includes or consists of a halosulfonic acid, for example chlorosulfonic acid, and the catalyst includes or consists of an aromatic amine, for example pyridine, or an amide, for example urea or dicyandiamide.

The invention also provides products of the processes described herein.

### DETAILED DESCRIPTION OF THE INVENTION

In some embodiments, the invention provides compounds having the Formula Ia or Ib: 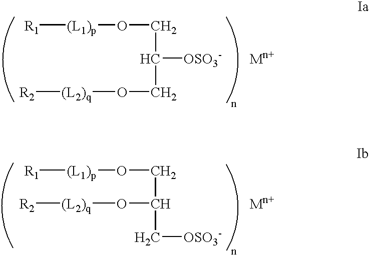
wherein:

M⁺ is a cation having charge n, for example an inorganic cation such as H⁺, Na⁺, K⁺ or NH₄⁺, or an organic cation such as primary, secondary, tertiary or quaternary ammonium, morpholinium or pyridinium;

n is 1, 2 or 3;

R₁ and R₂ are each independently C₁₋₂₅ alkyl, C₂₋₂₅ alkenyl, C₂₋₂₅ alkynyl, or C₂₋₂₅ polyunsaturated, each of which is optionally substituted with up to five substituents independently selected from the group consisting of halogen, optionally protected hydroxyl, C₁₋₁₂ alkoxy, C₁₋₂₄ alkoxyalkoxy, C₁₋₂₄ polyether, C₁₋₃ perhaloalkyl, C₁₋₃ perhaloalkoxy, or C₁₋₂₄ perhalopolyether;

L₁ and L₂ are each independently selected from the group consisting of —C(═O)— and —O—(C═O)—; and

p and q are each independently 0 or 1; provided that R₁ and R₂ together contain at least six carbon atoms.

In some embodiments, p and q are each 1. In some further embodiments, p and q are each 0. In some embodiments, L₁ and L₂ are each —C(═O)—. In some further embodiments, p and q are each 1; and L₁ and L₂ are each —C(═O)—.

In some embodiments, R₁ and R₂ are each independently C₃₋₂₁ alkyl, or C₅₋₂₁ alkyl, or C₃₋₁₇ alkyl. In some embodiments, p and q are each 1; L, and L₂ are each —C(═O); and R₁ and R₂ are each independently C₃₋₂₁ alkyl, or R₁ and R₂ are each independently C₅₋₂₁ alkyl, or R₁ and R₂ are each independently C₃₋₁₇ alkyl. In some embodiments, p and q are each 1; L₁ and L₂ are each —C(═O); and R₁ and R₂ are each n-C₉H₁₉, or R₁ and R₂ are each n-C₁₀H₂₁, or R₁ and R₂ are each n-C₁₁H₂₃, or R₁ and R₂ are each n-C₁₂H₂₅.

In a further aspect, the invention provides processes for the sulfation of a disubstituted glyceride, the process comprising reacting the disubstituted glyceride with a sulfating agent in the presence of a catalyst for a time and under conditions effective to form a disubstituted glyceride sulfate salt; wherein the sulfating agent includes or consists of sulfamic acid, or a halosulfonic acid.

In some embodiments, the sulfating agent includes or consists of sulfamic acid, and the catalyst includes or consists of a sulfamate salt, for example ammonium sulfamate. In other embodiments, the sulfating agent includes or consists of sulfamic acid, and the catalyst includes or consists of an amine, for example a secondary amine, for example a cyclic aliphatic amine optionally containing up to three heteroatoms selected from O, N and S, such as morpholine, or an amide, such as urea or dicyandiamide.

In some embodiments, the sulfating agent includes or consists of a halosulfonic acid, for example chlorosulfonic acid, and the catalyst includes or consists of an aromatic amine, for example pyridine, or an amide, for example urea or dicyandiamide.

In a further aspect, the present invention provides processes for the preparation of disubstituted glycerol sulfate salts, for example compounds having the Formula la or lb. It has been found in accordance with the present invention that disubstituted glycerol compounds, and their derivatives, can be easily sulfated by reaction with a sulfating agent that includes, or is composed of, sulfamic acid, preferably with a catalyst such as a sulfamate salt such as ammonium sulfamate, or an amide such as urea or dicyandiamide, or a secondary amine, for example a cyclic amine, such as morpholine. Thus, in some embodiments, the processes of the invention include:

a) providing a compound having the Formula IIa or IIb:

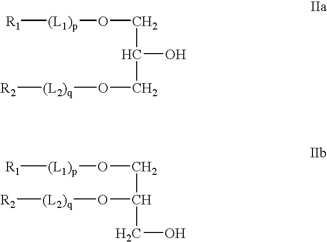

wherein the constituent variables are as defined above, and reacting the compound of Formula IIa or IIb with a sulfating agent in the presence of a catalyst for a time and under conditions effective to form the compound of Formula Ia or Ib as described above; wherein the sulfating agent comprises sulfamic acid.

In some embodiments, the catalyst includes or consists of a sulfamate salt. While a wide variety of sulfamate salts can be employed, in some preferred embodiments, the catalyst is ammonium sulfamate.

In some further embodiments, the catalyst includes or consists of an amine. Suitable amine catalysts include secondary amines, for example cyclic aliphatic amines that optionally containing up to three heteroatoms selected from O, N and S. One example of such an amine catalyst is morpholine. In some further embodiments, the catalyst includes or consists of an amide, such as urea or dicyandiamide.

The reaction of the compound of Formula IIa or IIb and the sulfamic acid sulfating agent can be performed at any effective and convenient temperature, although it is generally beneficial to conduct the reaction at elevated (i.e., above ambient) temperature. Generally, it is beneficial to perform the reaction at a temperature up to about 130° C., for example from about 90° C. to about 130° C., or from about 120° C. to about 130° C.

The elevated temperature is maintained for a time sufficient to provide an acceptable yield of product. Generally, the elevated temperature is maintained for a period of time that is about 30 minutes or longer, about one hour or longer, about two hours or longer, or about three hours or longer.

In some cases, it may be beneficial to remove some of the unreacted disubstituted glyceride from the reaction mixture after the reaction has completed. For example, the resulting reaction mixture containing product and unreacted disubstituted glycerides can optionally be cooled, and then optionally neutralized with alkali such as aqueous NaOH. The neutralized reaction mixture can then be diluted with water, and the unreacted disubstituted glyceride can be recovered, for example by centrifugation.

In some further embodiments, the invention provides process for the preparation of a compound having the Formula Ia or Ib: 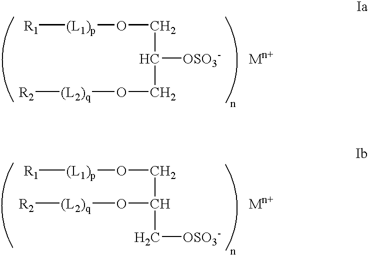
wherein the constituent variables are as defined above, which include:

a) providing a compound having the Formula IIa or IIb:

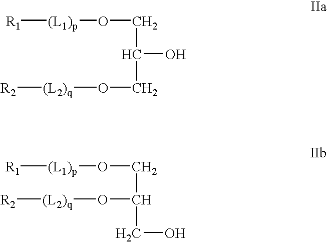

and reacting the compound of Formula IIa or IIb with a sulfating agent in the presence of a catalyst comprising an aromatic amine or an amide, for a time and under conditions effective to form the compound of Formula Ia or Ib; wherein the sulfating agent comprises a halosulfonic acid. One preferred halosulfonic acid is chlorosulfonic acid.

In some embodiments, the catalyst is an aromatic amine; i.e., an aromatic species that contains one or more ring nitrogen atoms. While not wishing to be bound by a particular theory, it is believed in accordance with the processes of the invention that the basic properties of the nitrogen atoms of such aromatic amines are particularly suited to facilitating the reaction between the halosulfonic acid and the disubstituted glycerol. One preferred aromatic amine is pyridine.

The reaction of the disubstituted glycerol (e.g., a compound of Formula IIa or IIb) with the halosulfonic acid (for example, chlorosulfonic acid) can be performed by any standard technique. For example, the reaction can be conveniently performed by first combining chlorosulfonic acid with pyridine to form a first solution or mixture; and then reacting the first solution or mixture with the disubstituted glycerol, for example a compound of Formula IIa or IIb.

Generally, the reaction of the compound of Formula IIa or IIb and the chlorosulfonic acid is performed for 30 minutes or longer, for example up to one hour, up to two hours, up to three hours, or up to about 5 hours, or longer. Products can be isolated by a variety of standard techniques known to the skilled artisan.

The product of the reaction may be isolated by various techniques known in the art. For example, in some cases it might be preferable to isolate the reaction product by extraction with an appropriate solvent or mixture of solvents, for example diethyl ether, and subsequent chromatography. Alternatively, it might be preferable in some cases to directly collect the product. For example, after the reaction of the disubstituted glycerol and the halosulfonic acid is complete, the pyridine can be removed (e.g., under reduced pressure; a nonpolar solvent (for example a hydrocarbon solvent such as hexane) can be added; any insoluble salt can be removed (for example by centrifugation); and the product can be extracted into an aqueous basic solution, for example an aqueous solution of a metal hydroxide, for example NaOH, preferably about 10% NaOH. In some cases it may be beneficial to further extract the product into a suitable solvent such as 1-butanol, and then remove the solvent to provide the sulfate salt of the disubstituted glyceride.

Alternatively, after the reaction of the disubstituted glycerol and the halosulfonic acid is complete, the pyridine can be removed under reduced pressure; a nonpolar solvent can be added, and the resulting mixture agitated (for example by swirling or shaking); the nonpolar solvent can be removed, and the product can be dissolved or dispersed in an aqueous basic solution having a pH of approximately 8 to form a solution or mixture which is then extracted with a suitable solvent such as 1-butanol; and the solvent can be removed to provide a salt of the disubstituted glyceride.

Generally; the reaction of the compound of Formula IIa or IIb and the chlorosulfonic acid is performed at a temperature of from about 40° C. to about 70° C., more preferably from about 50° C. to about 65° C.

In some embodiments, compound of Formula IIa or IIb is reacted with a halosulfonic acid sulfating agent in the presence of a catalyst that comprises an amide. Suitable amides include urea and dicyandiamide. In some embodiments, the reaction is conveniently performed by adding the halosulfonic acid, for example chlorosulfonic acid, to a dispersion of urea in a solvent, e.g., dry chloroform, and then reacting the dispersion with the disubstituted glycerol. For example, chlorosulfonic acid can be added to a dispersion of a suitable amide (e.g., urea) in a small volume of solvent, (such as dry chloroform), preferably maintaining the temperature below 30° C. The mixture is then heated (e.g., to 55-60° C.) and the disubstituted glyceride can be added to the mixture. Beneficially, the disubstituted glyceride can be added as a solution or dispersion in a small volume of dry chloroform. The mixture can then be stirred and heated for an additional period of time (for example one additional hour) after completing the addition of the diglyceride solution. After completion of the reaction, any solvent can be removed (for example by heating at about 60° C. under moderate vacuum), then the residue can be dissolved or dispersed in distilled water and neutralized to a pH of about 8 with alkali, for example NaOH, and unreacted disubstituted glyceride can be recovered, for example by centrifugation.

While not wishing to be bound by a particular theory, it is believed that the reaction of the disubstituted glycerol and chlorosulfonic acid in the presence of the amide (e.g., urea) produces the disubstituted glyceride sulfate in acid form. Because the acid form is believed to be less stable than the corresponding salt, it is believed to be beneficial to neutralize the reaction product as described above.

The reaction of the disubstituted glycerol and the halosulfonic acid can be performed at any effective and convenient temperature, although it is generally beneficial to conduct the reaction at elevated (i.e., above ambient) temperature. Generally, the reaction temperature is performed at a temperature of greater than about 30° C., for example from about 40° C. to about 60° C.

The elevated temperature is maintained for a time sufficient to provide an acceptable yield of product. Generally, the elevated temperature is maintained for a period of time that is about 30 minutes or longer, about one hour or longer, about two hours or longer, about three hours or longer, or about five hours or longer. In some embodiments, the reaction is performed for about 3 to about 5 hours at a temperature of from about 40° C. to about 60° C.

In some embodiments of each of the processes described herein, the product can be further purified by recrystallization. The recrystallization can be performed with a solvent, or with a mixture of solvents. In some embodiments, the product can be further purified by chromatography, for example on silica gel. Suitable elution solvents include halogenated hydrocarbons, for example methylene chloride. Other suitable solvents will be apparent to those of skill in the art. The isolated product may be further purified by washing one or more times with an appropriate solvent, or mixture of solvents.

Generally, in the processes of the invention, the reaction can be performed with equimolar quantities of sulfating agent and disubstituted glycerol (for example, a compound of Formula IIa or IIb). However, it is generally beneficial to employ the sulfating agent in molar excess relative to the disubstituted glycerol. For example, the sulfating agent can be employed in a molar excess of up to about 100%, or up to about 40%, or up to about 20%, or up to about 10%, relative to the amount of disubstituted glycerol. In some preferred embodiments, the sulfating agent is employed in a molar excess of up to about 10%, relative to the amount of disubstituted glycerol.

The catalyst can be employed in any convenient amount sufficient to provide an acceptable yield of product. For example, the catalyst can be employed in an amount that is about 1 mole percent, about 2 mole percent, about 5 mole percent, about 10 mole percent, about 15 mole percent, about 25 mole percent, about 50 mole percent, about 60 mole percent, about 100 mole percent, or more, relative to the amount of disubstituted glycerol.

In some embodiments, the reactions of the processes described herein can be carried out in the absence of a solvent. For example, where the disubstituted glycerol is a liquid at the desired reaction temperature, the disubstituted glycerol, sulfating agent (for example sulfamic acid) and catalyst reactants can be heated together without a solvent. Similarly, where halosulfonic acid sulfating agents are employed with an aromatic amine, for example pyridine, the amine can act as the solvent for the reaction. However, in some embodiments, such as the sulfation of disubstituted glycerides by chlorosulfonic acid with urea catalyst described above, it may be desirable to employ a solvent. Suitable solvents should be substantially nonreactive with the starting materials (reactants), the intermediates, or products at the temperatures at which the reactions are carried out, and can be readily selected by one of skill in the art of organic synthesis. Some suitable solvents include halogenated hydrocarbons such as chloroform and dichloromethane.

The product of the reaction may be isolated by various techniques known in the art. For example, in some cases it might be preferable to isolate the reaction product by extraction with an appropriate solvent or mixture of solvents, for example diethyl ether, and subsequent chromatography, or by centrifugation of an aqueous solution of the reaction product to separate and recover unreacted disubstituted glyceride. Alternatively, it might be preferable in some cases to directly collect the product.

In some embodiments, a mixture of compounds of Formula IIa and IIb can be employed as staring materials. In such cases, the mixture of sulfated products can be separated by standard techniques, for example those described herein.

In some embodiments, the processes described herein are used to prepare compounds of Formula I wherein p and q are each 1. In some further embodiments, p and q are each 0. In some embodiments, L₁ and L₂ are each —C(═O)—. In some further embodiments, p and q are each 1; and L₁ and L₂ are each —C(═O)—.

In some embodiments, the processes described herein are used to prepare compounds of Formula I wherein p and q are each 1; L₁ and L₂ are each —C(═O)—; and R₁ and R₂ are each independently C₃₋₂₁ alkyl, or R₁ and R₂ are each independently C₅₋₂₁ alkyl, or R₁ and R₂ are each independently C₃₋₁₇ alkyl. In some such embodiments, p and q are each 1; L₁and L₂ are each —C(═O)—R₁; and R₁ and R₂ are each n-C₉H₁₉, or R₁ and R₂ are each n-C₁₀H₂₁, or R₁ and R₂ are each n-C₁₁H₂₃, or R₁ and R₂ are each n-C₁₂H₂₅.

In the synthesis of many compounds described herein, protecting groups can be employed to protect various functionality or functionalities during the synthesis (for example, hydroxyl protecting groups can be used to protect hydroxyl groups appended to R₁ and R₂ groups). Representative protecting groups suitable for a wide variety of synthetic transformations are disclosed in Greene and Wuts, Protective Groups in Organic Synthesis, 2d ed, John Wiley & Sons, New York, 1991, the disclosure of which is incorporated herein by reference in its entirety.

The present invention also provides products of the processes described herein.

The processes of the invention provide product compounds of Formula I in yields of greater than about 50%, about 60%, about 70%, about 80%, about 90%, about 95%, about 96%, about 97%, about 98%, about 99%, or substantially about 100%.

In the Formulas Ia and Ib, the term M is a cation having charge n. When M is H⁺, the disubstituted glyceride sulfate compound is the free acid. Salts of the disubstituted glyceride sulfate compounds described herein include those formed from inorganic cations such as Na⁺, K⁺ or NH₄⁺, or other cations having greater charge (e.g., other metal cations), or organic cations such as primary, secondary, tertiary or quaternary ammonium, morpholinium or pyridinium ion.

As used herein, the term “alkyl” is meant to refer to a saturated hydrocarbon group which is straight-chained or branched. Examples of alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl), hexyl, octyl, decyl, dodecyl, and the like.

As used herein, the term “alkenyl” is meant to refer to an alkyl group that contains one or more carbon-carbon double bonds.

As used herein, the term “alkynyl” is meant to refer to an alkyl group that contains one or more carbon-carbon triple bonds.

As used herein, the term “polyunsaturated” is meant to refer to an alkyl group that contains at least one carbon-carbon double bond, and at least one carbon-carbon triple bond.

As used herein, “halo” or “halogen” refers to fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “alkoxyalkoxy” refers to an —O-alkyl-O-alkyl group.

As used herein, “polyether” refers to a group of formula —(O-alkyl)ₙ, where n is from 3 to 20.

The term “disubstituted glyceride” is intended to mean a glycerol molecule in which the hydrogen atoms of two of the hydroxyl groups attached to the carbon atoms of the glycerol have been replaced with a non-hydrogen substituent. Examples of such substituents include but are not limited to groups of formula -(L₁)ₚ—R₁ and -(L₂)_{q}—R₂ as described herein.

As used herein, the term “reacting” refers to the bringing together of designated chemical reactants such that a chemical transformation takes place generating a compound different from any initially introduced into the system. Reacting can take place in the presence or absence of solvent.

As used herein, the term “contacting” means the placing together of the indicated components such that a mixture or solution is formed.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl and C₆ alkyl.

The compounds of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

The processes described herein can be monitored according to any suitable process known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography. Such techniques can be used to detect the disappearance of starting material (e.g. compound of Formula II), or detect the appearance of product, or both.

The reactions of the processes described herein can be carried out in air or under an inert atmosphere. Typically, reactions containing reagents or products that are substantially reactive with air can be carried out using air-sensitive synthetic techniques that are well known to the skilled artisan.

The surfactant compounds of the invention are useful in a variety of settings. For example, the compounds are useful as components of detergents, emulsifiers, paints, adhesives, inks, and as wetting agents in coating for various substrates including window washing compounds, floor polishes and finishes, cosmetics, and electronic component cleaners. They are also useful in pharmaceutical preparations, as cement additives, and in the enhanced recovery of oil from petroleum reservoirs. In addition, the compounds of the invention possess excellent foaming properties, and are thus excellent foaming agents. Accordingly, they are useful as components of shampoos, and hand dishwashing products.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The processes of this invention are suitable for the preparation of compounds Formula I on any convenient scale, for example greater than about 0.01 mg, 0.10 mg, 1 mg, 10 mg, 100 mg, 1 g, 10 g, 100 g, 1 kg, 10 kg or more. The processes are particularly advantageous for the large scale (e.g., greater than about ten gram) and very large scale (e.g., greater than about 1 kilogram) preparation of compounds of the invention.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of the Ammonium or Sodium Salt of Sulfated Dioctanoyl Glyceride

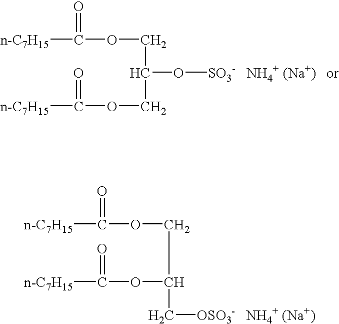

Dioctanoyl glyceride (6.88 grams; 0.02 mole), sulfamic acid (2.14 gram; 0.022 mole) and ammonium sulfamate (0.34 gram; 0.00298 mole) were heated with stirring at 120-125° C. for 3 hours. The reaction mixture was a somewhat viscous brown liquid. The reaction mixture was treated with water to make a 10% solution by weight, and centrifuged to recover unreacted diglyceride. When diluted to make a 0.1% by weight solution of the reaction mixture, the aqueous solution was slightly translucent, and had a surface tension at 25° C. of 25.4 mN/m. 20 ml of the 0.1% solution, when shaken in a 100 ml graduated cylinder for 20 full cycles, produced 45 ml of foam that persisted for one hour or more.

Alternatively, the reaction mixture was treated an aqueous solution containing 0,025 moles of sodium hydroxide to form a 10% by weight solution of the reaction mixture, and centrifuged to recover unreacted diglyceride. When diluted to make a 0.1% by weight solution of the reaction mixture, the aqueous solution had a surface tension at 25° C. of 26.3 mN/m.

### Example 2

### Preparation of the Ammonium Salt of Sulfated Dihexanoyl Glyceride

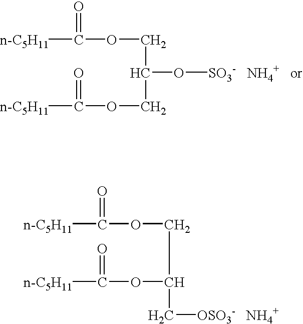

Dihexanoyl glyceride (5.76 grams; 0.02 mole), sulfamic acid (2.14 gram; 0.022 mole) and ammonium sulfamate (0.34 gram; 0.00298 mole) were heated with stirring at 120-124° C. for 1.5 hours. The reaction mixture was treated with water to make a 10% solution by weight, and centrifuged to recover unreacted diglyceride. When diluted to make a 0.4% by weight solution of the reaction mixture, the aqueous solution had a surface tension at 25° C. of 31.9 mN/m.

### Example 3

### Preparation of the Mixed Morpholinium, Ammonium Salt of Sulfated Dihexanoyl Glyceride

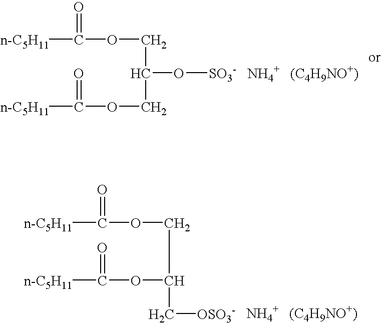

Dihexanoyl glyceride (5.76 grams; 0.02 mole), sulfamic acid (2.14 gram; 0.022 mole) and morpholine (1 ml, 0.011 mole) were heated with stirring at 120-125° C. for 6 hours. The solid sulfamic acid became dispersed in the reaction product as heating proceeded. After 6 hours of heating, a sample of the reaction mixture was taken. The reaction product was treated with water to make a 10% solution by weight and centrifuged to recover unreacted diglyceride. When diluted to make a 0.44% solution of the reaction mixture, the aqueous solution had a surface tension at 25° C. of 36.5 mN/m.

### Example 4

### Synthesis of Sodium Salt of Sulfonated Didecanoyl Glyceride

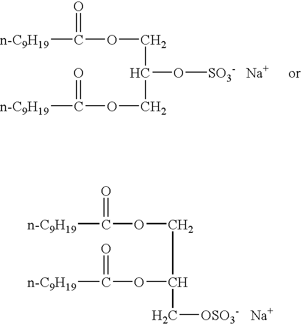

2.4 g urea (0.04 moles) was dispersed in 10 ml dry CHCl₃. Then, 2.6 ml of chlorosulfonic acid (4.64 g, 0.04 moles) was added, dropwise, to the stirred urea-CHCl₃ dispersion, maintaining the temperature below 30° C. The mixture was heated to 55-60° C. and 8.0 g (0.02 moles) of didecanoyl glyceride was added dropwise to the stirred chlorsulfonic acid-urea-chloroform mixture at 55-60° C. for a period of about 45 minutes (the didecanoyl glyceride is dissolved in 15 ml dry CHCl₃ before the addition). The reaction mixture became clear when about two thirds of the didecanoyl glyceride in chloroform solution had been added. The mixture was stirred and heated for one additional hour at 60° C. after completing the addition of the diglyceride solution.

A sample of the reaction mixture, whose weight was 0.1 g after removal of the chloroform therefrom at 60° C. under moderate vacuum (200 mm Hg), was dissolved in distilled water and neutralized to a pH of about 8 with NaOH. The solution had a surface tension at 25° C. of 25.9 mN/m.

The reaction mixture was heated to 60° C. at 30 mm pressure to remove chloroform. The residue (an off-white viscous liquid) was dissolved in water and neutralized to a pH of about 8 to convert it to the sodium salt. Unreacted diglyceride could be removed by centrifugation of the aqueous solution.

### Example 5

### Preparation of the Sodium Salt of Sulfated Dioctanoyl Glyceride

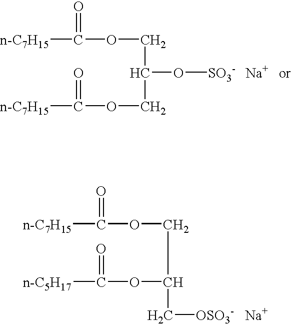

To 20 ml of dry pyridine in a 3-neck flask, under anhydrous conditions, was added 4 ml (0.0614 mole) of chlorosulfonic acid dropwise, with stirring and cooling, maintaining the temperature of the mixture below 30° C. (with an ice bath). After the addition of the chlorosulfonic acid was complete, the reaction mixture was heated to 50° C. to 60° C., and 10.6 grams (0.031 mole) of dioctanoyl glyceride was added dropwise, stirring the reaction mixture, for a period of 4.5 hours. The excess pyridine was removed at 60° C. and 20 mm pressure. 50 ml of hexane was added, and the mixture was shaken well. The hexane layer, which contains unreacted dioctanoyl glyceride, (about 20% of the original amount) is removed.

To the hexane-extracted reaction mixture is added 50 ml of water and 10% aqueous NaOH solution until the pH is approximately 8. 40 ml of butanol was added, and the mixture was shaken well. The 1-butanol layer was collected and the butanol evaporated at 60° C. and 20 mm pressure to yield the product as an off-white, viscous semi-solid.

A 0.12% aqueous solution of the product in distilled water had a surface tension at 25° C. of 26.8 mN/m, and was observed to foam very well.

### Example 6

### Preparation of the Ammonium Salt of Sulfated Dihexanoyl Glyceride and Preparation of Mixed Sodium and Ammonium Salt of Sulfated Dihexanoyl Glyceride

I. Ammonium Salt:

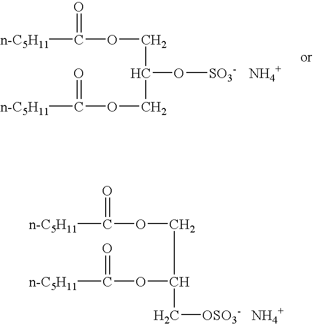

Dihexanoyl glyceride (5.76 6 g, 0.02 moles) was heated with 2.14 g (0.022 moles) of sulfamic acid and 0.66 g (0.011 moles) of urea for 2.5 hours at 120° C.

A 0.42% aqueous solution of the product had a surface tension at 25° C. of 35.6 mN/m.
II. Mixed Ammonium/Sodium Salt:

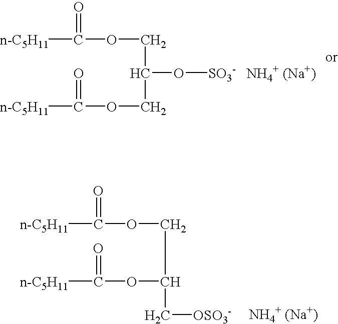

10% NaOH was added to the reaction mixture from I above, and the mixture was diluted with water to make a solution containing 10% by weight of the reaction mixture, adjusted to a pH of about 9. The mixture, which emulsified when the water was added, was then centrifuged to recover some of the unreacted diglyceride.

A 0.42% (by weight) solution of the reaction mixture (made by diluting the 10% solution after centrifugation) had a surface tension at 25° C. of 35.0 mN/m.

As those skilled in the art will appreciate, numerous changes and modifications may be made to the preferred embodiments of the invention without departing from the spirit of the invention. It is intended that all such variations fall within the scope of the invention. It is intended that each of the patents, applications, and printed publications including books mentioned in this patent document be hereby incorporated by reference in their entirety.

What is claimed is:

1. A compound having the Formula Ia or Ib: 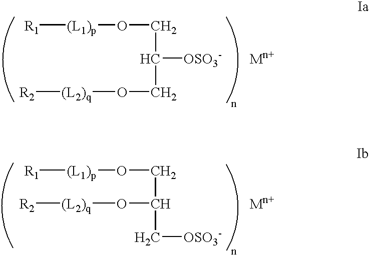
wherein:
M is a cation having charge n⁺;
n is 1, 2 or 3;
R₁ and R₂ are each independently C₁₋₂₅ alkyl, C₂₋₂₅ alkenyl, C₂₋₂₅ alkynyl, or C₂₋₂₅ polyunsaturated, each of which is optionally substituted with up to five substituents independently selected from the group consisting of halogen, optionally protected hydroxyl, C₁₋₁₂ alkoxy, C₁₋₂₄ alkoxyalkoxy, C₁₋₂₄ polyether, C₁₋₃ perhaloalkyl, C₁₋₃ perhaloalkoxy, or C₁₋₂₄ perhalopolyether;
L₁ and L₂ are each independently selected from the group consisting of —C(═O)— and —O—(C═O)—; and
p and q are each independently 0 or 1;
provided that R₁ and R₂ together contain at least six carbon atoms.

2. The compound of claim 1 wherein p and q are each 1.

3. The compound of claim 1 wherein p and q are each 0.

4. The compound of claim 1 wherein L₁ and L₂ are each —C(═O)—.

5. The compound of claim 1 wherein p and q are each 1; and L₁ and L₂ are each —C(═O)—.

6. The compound of claim 5 wherein R₁ and R₂ are each independently C₃₋₂₁ alkyl.

7. The compound of claim 5 wherein R₁ and R₂ are each independently C₅₋₂₁ alkyl.

8. The compound of claim 5 wherein R₁ and R₂ are each independently C₃₋₁₇ alkyl.

9. The compound of claim 5 wherein R₁ and R₂ are each n-C_{g}H₁₉.

10. The compound of claim 5 wherein R₁ and R₂ are each n-C₁₀H₂₁.

11. The compound of claim 5 wherein R₁ and R₂ are each n-C₁₁H₂₃.

12. The compound of claim 5 wherein R₁ and R₂ are each n-C₁₂H₂₅.

13. A process for the preparation of a compound having the Formula Ia or Ib: 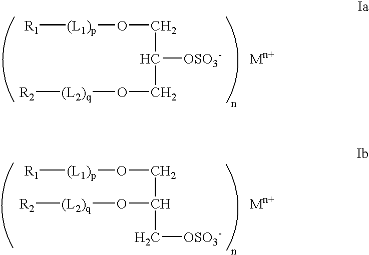
wherein:
M is a cation having charge n⁺;
n is 1, 2 or 3;
R₁ and R₂ are each independently C₁₋₂₅ alkyl, C₂₋₂₅ alkenyl, C₂₋₂₅ alkynyl, or C₂₋₂₅ polyunsaturated, each of which is optionally substituted with up to five substituents independently selected from the group consisting of halogen, optionally protected hydroxyl, C₁₋₁₂ alkoxy, C₁₋₂₄ alkoxyalkoxy, C₁₋₂₄ polyether, C₁₋₃ perhaloalkyl, C₁₋₃ perhaloalkoxy, or C₁₋₂₄ perhalopolyether;
L₁ and L₂ are each independently selected from the group consisting of —C(═O)— and —O—(C═O)—; and
p and q are each independently 0 or 1;
provided that R₁ and R₂ together contain at least six carbon atoms;
comprising:
a) providing a compound having the Formula IIa or IIb: 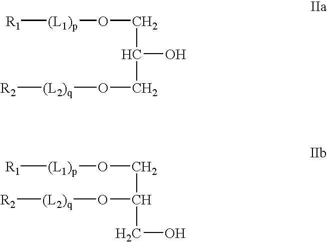
and reacting the compound of Formula IIa or IIb with a sulfating agent in the presence of a catalyst for a time and under conditions effective to form the compound of Formula Ia or Ib;
wherein the sulfating agent comprises sulfamic acid.

14. The process of claim 13 wherein the catalyst comprises a sulfamate salt.

15. The process of claim 13 wherein the catalyst comprises ammonium sulfamate.

16. The process of claim 13 wherein the catalyst comprises an amine.

17. The process of claim 13 wherein the catalyst comprises a secondary amine.

18. The process of claim 13 wherein the catalyst comprises a cyclic aliphatic amine optionally containing up to three heteroatoms selected from O, N and S.

19. The process of claim 13 wherein the catalyst comprises morpholine.

20. The process of claim 13 wherein the catalyst comprises an amide.

21. The process of claim 13 wherein the catalyst comprises urea or dicyandiamide.

22. The process of claim 13 wherein p and q are each 1.

23. The process of claim 13 wherein p and q are each 0.

24. The process of claim 13 wherein L₁ and L₂ are each —C(═O)—.

25. The process of claim 13 wherein p and q are each 1; and L₁ and L₂ are each —C(═O)—.

26. The process of claim 14 wherein p and q are each 1; and L₁ and L₂ are each —C(═O)—.

27. The process of claim 26 wherein R₁ and R₂ are each independently C₃₋₂₁ alkyl.

28. The process of claim 26 wherein R₁ and R₂ are each independently C₅₋₂₁ alkyl.

29. The process of claim 26 wherein R₁ and R₂ are each independently C₃₋₁₇ alkyl.

30. The process of claim 13 wherein the reaction of the compound of Formula IIa or IIb and the sulfating agent comprises heating a solution or mixture containing the compound of Formula IIa or IIb and the sulfating agent at a temperature of from about 90° C. to about 130° C.

31. The process of claim 30 wherein the heating is performed for about 30 minutes or longer.

32. The process of claim 13 further comprising the steps of:
(i) heating the compound of Formula IIa or IIb with the sulfating agent in the presence of the catalyst to form a reaction mixture comprising the compound of Formula Ia or Ib;
(ii) optionally cooling the reaction mixture;
(iii) optionally neutralizing the reaction mixture with alkali;
(iv) optionally diluting the reaction mixture with water; and
(v) recovering unreacted compound of Formula IIa and IIb.

33. The process of claim 32 wherein the unreacted compound of Formula IIa and IIb is recovered by centrifugation.

34. The process of claim 13 wherein the sulfamic acid is present in molar excess relative to the compound of Formula II.

35. The process of claim 13 wherein the sulfamic acid is present in an amount that is up to about 20% molar excess relative to the compound of Formula IIa or IIb.

36. The process of claim 13 wherein the catalyst is present in an amount that is up to about 100 mole percent relative to the amount of the compound of Formula IIa or IIb.

37. A product prepared by the process of claim 13.

38. A process for the preparation of a compound having the Formula Ia or Ib: 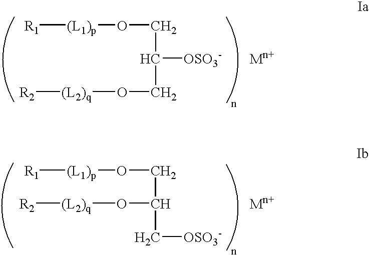
wherein:
M is a cation having charge n⁺;
n is 1, 2 or 3;
R₁ and R₂ are each independently C₁₋₂₅ alkyl, C₂₋₂₅ alkenyl, C₂₋₂₅ alkynyl, or C₂₋₂₅ polyunsaturated, each of which is optionally substituted with up to five substituents independently selected from the group consisting of halogen, optionally protected hydroxyl, C₁₋₁₂ alkoxy, C₁₋₂₄ alkoxyalkoxy, C₁₋₂₄ polyether, C₁₋₃ perhaloalkyl, C₁₋₃ perhaloalkoxy, or C₁₋₂₄ perhalopolyether;
L₁ and L₂ are each independently selected from the group consisting of —C(═O)— and —O—(C═O)—; and
p and q are each independently 0 or 1;
provided that R₁ and R₂ together contain at least six carbon atoms;
comprising:
a) providing a compound having the Formula IIa or IIb: 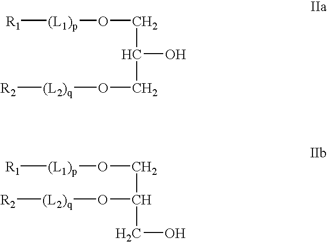
and reacting the compound of Formula IIa or IIb with a sulfating agent in the presence of a catalyst comprising an aromatic amine or an amide for a time and under conditions effective to form the compound of Formula Ia or Ib;
wherein the sulfating agent is a halosulfonic acid.

39. The process of claim 38 wherein the sulfating agent is chlorosulfonic acid.

40. The process of claim 38 wherein the sulfating agent is chlorosulfonic acid, and the catalyst is an aromatic amine.

41. The process of claim 38 wherein the sulfating agent is chlorosulfonic acid, and the catalyst is pyridine.

42. The process of claim 41 wherein the reaction of the compound of Formula IIa or IIb with chlorosulfonic acid is performed by the steps of:
a) combining the chlorosulfonic acid with pyridine to form a first solution or mixture; and
b) reacting the first solution or mixture with the compound of Formula II.

43. The process of claim 41 wherein the reaction of the compound of Formula IIa or IIb and the chlorosulfonic acid is performed at a temperature of from about 40° C. to about 80° C.

44. The process of claim 38 wherein the sulfating agent is chlorosulfonic acid, and the catalyst is an amide.

45. The process of claim 38 wherein the sulfating agent is chlorosulfonic acid, and the catalyst is urea.

46. The process of claim 45 wherein the reaction of the compound of Formula IIa or IIb with chlorosulfonic acid is performed by the steps of:
(a) combining the chlorosulfonic acid with the urea in a solvent to form a first solution or mixture; and
(b) reacting the first solution or mixture with the compound of Formula II.

47. The process of claim 45 wherein the reaction of the compound of Formula IIa or IIb and the chlorosulfonic acid is performed at a temperature of from about 40° C. to about 60° C.

48. The process of claim 46 further comprising:
(c) removing the solvent to provide a residue; and
(d) contacting the residue with an aqueous alkali solution to form a neutralized solution or dispersion.

49. The process of claim 48 wherein the solvent is removed by heating under a vacuum.

50. The process of claim 48 further comprising:
e) separating unreacted disubstituted glyceride from the neutralized solution or dispersion.

51. The process of claim 50 wherein the unreacted disubstituted glyceride is separated from the neutralized solution or dispersion by centrifugation.

52. A product of the process of claim 38.

53. A process for the sulfation of a disubstituted glyceride, the process comprising reacting the disubstituted glyceride with a sulfating agent in the presence of a catalyst for a time and under conditions effective to form a disubstituted glyceride sulfate, or salt thereof; wherein the sulfating agent comprises sulfamic acid or a halosulfonic acid.

54. The process of claim 53 wherein the sulfating agent comprises sulfamic acid.

55. The process of claim 54 wherein the catalyst comprises a sulfamate salt.

56. The process of claim 54 wherein the catalyst comprises ammonium sulfamate.

57. The process of claim 54 wherein the catalyst comprises an amine.

58. The process of claim 54 wherein the catalyst comprises a secondary amine.

59. The process of claim 54 wherein the catalyst comprises a cyclic aliphatic amine optionally containing up to three heteroatoms selected from O, N and S.

60. The process of claim 54 wherein the catalyst comprises morpholine.

61. The process of claim 54 wherein the catalyst comprises an amide.

62. The process of claim 54 wherein the catalyst comprises urea of dicyandiamide.

63. The process of claim 53 wherein the sulfating agent comprises chlorosulfonic acid.

64. The process of claim 53 wherein the sulfating agent comprises chlorosulfonic acid, and the catalyst is an aromatic amine.

65. The process of claim 53 wherein the sulfating agent comprises chlorosulfonic acid, and the catalyst is pyridine.

66. The process of claim 53 wherein the sulfating agent comprises chlorosulfonic acid, and the catalyst is an amide.

67. The process of claim 53 wherein the sulfating agent comprises chlorosulfonic acid, and the catalyst is urea or dicyandiamide.

68. A composition comprising a compound of claim 1.
